(19) United States
(12) Patent Application Publication
(10) Pub. No.: US 2021/0113638 A1
(43) Pub. Date: Apr. 22, 2021

(54) PLANT-BASED NERIUM OLEANDER EXTRACTS AND METHODS

(71) Applicant: NERIUM BIOTECHNOLOGY, INC., San Antonio, TX (US)

(72) Inventors: Dennis Ray Knocke, San Antonio, TX (US); Joseph Benjamin Nester, San Antonio, TX (US)

(21) Appl. No.: 17/044,781

(22) PCT Filed: Mar. 28, 2019
(86) PCT No.: PCT/US2019/024652

§ 371 Date: Oct. 1, 2020

Related U.S. Application Data
(60) Provisional application No. 62/651,584, filed on Apr. 2, 2018.

(51) Int. Cl. A61K 36/24; A61K 47/44; A61K 47/46; A61K 8/9789; A61Q 19/08; A61K 9/00; A61K 8/34; A61K 8/86; A61K 8/891; A61K 8/73; A61K 8/37; A61K 8/67; A61K 47/12; A61K 47/10; A61K 47/06; A61K 47/26; A61K 47/36; A61K 47/14

(52) U.S. Cl. CPC A61K 36/24; A61K 2236/37; A61K 47/46; A61K 8/9789; A61Q 19/08; A61K 9/0014; A61K 8/345; A61K 8/86; A61K 8/342; A61K 8/891; A61K 8/731; A61K 8/37; A61K 8/678; A61K 47/12; A61K 47/10; A61K 47/06; A61K 47/26; A61K 47/36; A61K 47/14; A61K 2236/333; A61K 2236/31; A61K 47/44

(57) ABSTRACT

Methods of extracting Nerium oleander plant species with a plant juice or plant oil are disclosed. Pharmaceutical and cosmetic compositions comprising the resulting Nerium oleander extracts and methods of using them are also disclosed.

### INCORPORATION BY REFERENCE TO ANY PRIORITY APPLICATIONS

This application claims priority to U.S. Provisional Application 62/651,584 filed Apr. 2, 2018. Any and all applications for which a foreign or domestic priority claim is identified in the Application Data Sheet as filed with the present application are hereby incorporated by reference under 37 CFR 1.57.

### BACKGROUND

### Field

This disclosure relates to methods of extracting Nerium oleander plant material and compositions comprising the resulting extraction products. Specifically, it relates to methods of extracting Nerium oleander plant material with the natural juice or oil of other plants.

### Description of the Related Art

Oleandrin is a cardiac glycoside having the following chemical structure:

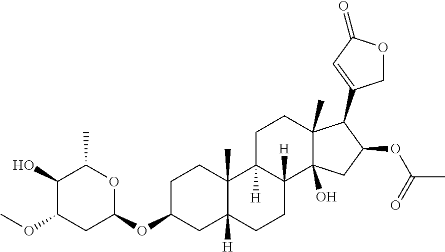

Oleandrin inhibits Na—K ATPase and has multiple medicinal and therapeutic uses. For example, U.S. Patent Publication Nos. 2005/0026849 and 2006/0205679 disclose oleandrin-containing formulations for use in treating various cell proliferative and inflammatory conditions.

Oleandrin can be obtained by extraction from the Nerium oleander plant species. For example, U.S. Pat. No. 5,135,745 discloses oleandrin-containing extracts obtained by extracting Nerium oleander plant material with hot water; U.S. Pat. No. 7,402,325 discloses oleandrin-containing extracts obtained by extracting Nerium oleander plant material with supercritical carbon dioxide, and U.S. Pat. No. 8,524,286 discloses oleandrin-containing extracts obtained by extracting Nerium oleander plant material with aloe.

### SUMMARY

Some embodiments relate to a method of extracting Nerium oleander, comprising:

providing an extractant liquid that comprises a plant juice or plant oil;

intermixing a Nerium oleander plant species with the extractant liquid under conditions selected to form an extraction mixture;

conditioning the extraction mixture under conditions selected to extract the Nerium oleander plant species to thereby form a conditioned extraction mixture, wherein the conditioned extraction mixture comprises solid residual Nerium oleander plant species and a liquid Nerium oleander extract; and

separating at least a portion of the liquid Nerium oleander extract from the solid residual Nerium oleander plant species to thereby form a separated liquid Nerium oleander extract;

wherein the extractant liquid does not consist of aloe.

Some embodiments relate to a liquid Nerium oleander extract prepared by a method as described herein.

Some embodiments relate to a pharmaceutical composition, comprising a Nerium oleander extract prepared according to a method as described herein, and a pharmaceutically acceptable carrier.

Some embodiments relate to a cosmetic composition, comprising a Nerium oleander extract prepared according to a method as described herein, and a topically acceptable carrier.

Some embodiments relate to a method of treatment, comprising identifying a subject having a skin condition; and applying an effective amount of a pharmaceutical composition as described herein to the skin of the subject to thereby treat the skin condition.

Some embodiments relate to a method of applying a cosmetic, comprising identifying a subject in need of cosmetic application; and applying a cosmetically effective amount of a cosmetic composition as described herein to the skin of the subject.

These and other embodiments are described in greater detail below.

### DETAILED DESCRIPTION

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The term “Nerium oleander” as used herein has its ordinary meaning as understood by those skilled in the art and thus includes the Nerium oleander species of plant. This plant is widespread and known by a number of synonyms in various places. Thus Nerium oleander also refers to the plants known as Oleander, Nerion, Nerion oleandrum, Nerium carneum, Nerium flavescens, Nerium floridum, Nerium grandiflorum, Nerium indicum, Nerium japonicum, Nerium kotschyi, Nerium latifolium, Nerium lauriforme, Nerium luteum, Nerium madonii, Nerium mascatense, Nerium odoratissimum, Nerium odoratum, Nerium odorum, Nerium splendens, Nerium thyrsiflorum, Nerium verecundum, Oleander indica, and Oleander vulgaris.

The term “aloe” as used herein has its ordinary meaning as understood by those skilled in the art and thus refers to the juice obtained from the succulent plant species known as Aloe vera. This plant is widespread and known by a number of synonyms in various places. Thus “aloe” also refers to the juice obtained from plants known as Aloe barbadensis, Aloe indica, Aloe perfoliata, and Aloe vulgaris.

The term “plant juice” as used herein has its ordinary meaning as understood by those skilled in the art and thus refers to the natural juice obtained from a plant, including mixtures obtained from two or more plants. Plant juices contain water and various water-soluble plant constituents. For example, in various embodiments the plant juice is a citrus juice such as lemon juice, lime juice and/or orange juice. Non-limiting examples of plant juices include apple juice, cherry juice, cranberry juice, grape juice, grapefruit juice, lemon juice, lime juice, orange juice, pear juice, pineapple juice and mixtures of any two or more of the foregoing. Plant juices may be concentrates that are formed by removing water from the natural plant juice as obtained from the plant, or may be dilutions that are formed by adding water to the natural plant juice as obtained from the plant. The amount of such concentration or dilution can vary by a factor of about 10. Thus, a plant juice concentrate can have a weight that is in the range of from 10% to slightly below 100% of the weight of the natural plant juice from which it is formed, and a plant juice dilution can have a weight that is in the range of from slightly above the weight of the natural plant juice from which it is formed to 10 times the weight of the natural plant juice from which it is formed.

The term “plant oil” as used herein has its ordinary meaning as understood by those skilled in the art and thus refers to the natural oil obtained from a plant, including mixtures obtained from two or more plants. Plant oil generally contains various hydrophobic plant constituents having a solubility in water of less than 10% by weight, based on total weight. Non-limiting examples of plant oils include almond oil, avocado oil, banana oil, basil oil, canola oil, cocoa butter, corn oil, cotton seed oil, ginger oil, hemp oil, lavender oil, lemon oil, linseed oil, mint oil, neem oil, olive oil, orange oil, parsley oil, pecan oil, rose oil, safflower oil, sesame oil, tea oil, vanilla oil, walnut oil and mixtures of any two or more of the foregoing. A plant oil may be an herb oil. Non-limiting examples of herb oils include the plant oils obtained from plant species such as Carum petroselinum (e.g., parsley oil) and Ocimum basilicum (e.g., basil oil).

The term “skin care product” as used herein has its ordinary meaning as understood by those skilled in the art and thus includes manufactured products that are formulated for application to human skin. In the context of this disclosure a skin care product typically contains active ingredients such as a Nerium oleander extract or combination of extracts, along with a topically acceptable carrier for those active ingredients. Examples of skin care products include cosmetic compositions and pharmaceutical compositions as described elsewhere herein.

The term “cosmetic composition” as used herein has its ordinary meaning as understood by those skilled in the art and thus includes skin care products that are formulated for cosmetic applications. Cosmetic compositions may contain active ingredients such as a Nerium oleander extract or combination of extracts in amounts that are effective to provide a desired cosmetic treatment effect, but below the amounts that provide a medical treatment effect. Cosmetic compositions may also contain a cosmetically acceptable carrier for the active ingredients. The term “cosmetically acceptable carrier” as used herein has its ordinary meaning as understood by those skilled in the art and thus includes the topically acceptable carriers described elsewhere herein that are suitable for formulating cosmetic compositions.

The term “topically acceptable carrier” as used herein has its ordinary meaning as understood by those skilled in the art and thus includes various ingredients that are physiologically compatible with human skin and thus useful for formulating skin care products. Suitable topically acceptable carrier materials include any carrier or vehicle commonly used as a base for solutions, dispersions, emulsions, gels, creams, ointment, lotions, pastes, or foams, for topical administration. Examples include emulsifying agents, inert carriers including hydrocarbon bases, emulsifying bases, non-toxic solvents or water-soluble bases. Examples of topically acceptable carriers include cosmetically acceptable carriers and pharmaceutically acceptable carriers as described elsewhere herein.

A variety of suitable liquid or gel-based topically acceptable carriers are well-known in the art. The topically acceptable carrier should be able to dissolve or disperse the Nerium oleander extract or combination of extracts at an effective level, optionally with the aid of non-toxic surfactants. Examples include water, physiological salt solutions, alcohols (e.g., methanol, ethanol, propanol, or butanol), glycerol, glycols (e.g., ethylene glycol, propylene glycol, or ethoxy diglycol), polyethylene glycol (e.g., having a molecular weight (MW) in the range of 400 to 20,000), water-alcohol/glycol blends, and combinations thereof. Suitable topically acceptable carriers and diluents for certain embodiments include, for example, water, saline, isotonic saline solutions (for example, phosphate-buffered saline), aqueous dextrose, glycerol, ethoxy diglycol, dimethyl sulfoxide (DMSO), and combinations thereof.

Suitable topically acceptable carriers further include aqueous and oleaginous carriers such as, for example, white petrolatum, isopropyl myristate, lanolin or lanolin alcohols, mineral oil, fragrant or essential oil, nasturtium extract oil, sorbitan mono-oleate, cetostearyl alcohol (together or in various combinations), detergents (e.g., polysorbates (TWEEN) such as polysorbate 20, polysorbate 40, polysorbate 60, or polysorbate 80; polyoxyl stearate; or sodium lauryl sulfate), and combinations thereof. One or more topically acceptable carrier materials can be mixed with water to form a lotion, gel, cream, or semi-solid composition. Other suitable topically acceptable carriers include water-in-oil or oil-in-water emulsions and mixtures of emulsifiers and emollients with solvents such as sucrose stearate, sucrose cocoate, sucrose distearate, mineral oil, propylene glycol, 2-ethyl-1,3-hexanediol, polyoxypropylene-15-stearyl ether, water, or combinations thereof. For example, emulsions containing water, glycerol stearate, glycerin, mineral oil, synthetic spermaceti, cetyl alcohol, or combinations thereof, may be used. Preservatives may also be included in the topically acceptable carrier, such as one or more of butylparaben, methylparaben, propylparaben, benzyl alcohol, ethylene diamine tetraacetate salts, and combinations thereof. The composition of the carrier can be varied so long as it does not interfere significantly with the beneficial effects of the Nerium oleander extract or combination of extracts of the topically acceptable composition.

The topically acceptable carriers described herein can include one or more gelling agents to increase the viscosity of the resulting composition. Examples of gelling agents and thickening agents, include, but are not limited to, fatty acids, fatty acid salts and esters, fatty alcohols, synthetic polymers, modified celluloses, xanthan gum, or combinations thereof. Other examples of gelling and thickening agents include lanolin, hard paraffin, liquid paraffin, white petrolatum, soft yellow paraffin or soft white paraffin, white beeswax, yellow beeswax, propolis (propoleum), cetostearyl alcohol, cetyl alcohol, dimethicones, emulsifying waxes, microcrystalline wax, oleyl alcohol, stearyl alcohol and combinations thereof.

Examples of suitable synthetic polymers for use as gelling and thickening agents include polyethylene glycol (PEG), polyvinylpyrrolidone (PVP), polyvinyl alcohol (PVA), various poloxamers (e.g. PLURONIC poloxamers), or carbomers (e.g., CARBOPOL 940 or CARBOPOL 934, Lubrizol Advanced Materials Inc.). Examples of suitable modified celluloses include methylcellulose, carboxymethylcellulose (CMC), hydroxyethylcellulose (HEC), hydroxymethyl cellulose (HMC), hydroxypropyl cellulose (HPC), hydroxypropylmethylcellulose (HPMC), and combinations thereof.

A gelling agent or thickening agent can be present in a topically acceptable carrier as described herein in an amount in the range of about 0.05 wt. % to about 20 wt. %, typically about 0.1 wt. % to about 10 wt. %, about 0.1 wt. % to about 5 wt. %, about 0.5 wt. % to about 2 wt. %, about 0.8 wt. % to about 2 wt. %, or about 1.0 wt. % to about 1.5 wt. %. One or more gelling agents or thickening agents may be included in a single formulation. Such agents can be employed with liquid carriers to form ointments or creams for application directly to the skin of the user.

Topically acceptable carrier solutions of one or more Nerium oleander extracts can be prepared in water, optionally mixed with a nontoxic surfactant. Dispersions can be prepared in glycerol, liquid polyethylene glycols, triacetin, or in a topically acceptable oil such as emu oil, or mixtures thereof. Under ordinary conditions of storage and use, preparations may contain a preservative to inhibit or prevent the growth of microorganisms. Solutions can be prepared by incorporating the Nerium oleander extract(s) in a desired amount in the appropriate solvent or oil with various other ingredients enumerated herein, as desired, followed by optional filter sterilization. For powders used in the preparation of solutions, methods of preparation can include vacuum drying and freeze drying techniques, which yield a powder of the Nerium oleander extract plus any additional desired ingredient present in the prepared solutions.

Topically acceptable carrier creams are viscous liquids or semisolid emulsions, either oil-in-water or water-in-oil. Cream bases are water-washable, and comprise an oil phase, an emulsifier, and an aqueous-phase. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH. The oil phase of a cream is generally comprised of petrolatum and a fatty alcohol such as cetyl or stearyl alcohol. The aqueous phase usually, although not necessarily, exceeds the oil phase in volume, and generally contains a humectant (a substance, such as glycerin, sorbitol, or urea, that absorbs or helps another substance retain moisture).

The emulsifier in a cream formulation is generally a nonionic, anionic, cationic, or amphoteric surfactant. Examples of emulsifiers include, but are not limited to, fatty alcohol polyoxyethylene ether (Peregal A-20), stearates such as polyoxylstearate (Softener SG), glyceryl stearate and pegylated forms of glyceryl stearate such as PEG-5 glyceryl stearate, cetyl alcohol, dithranol, or a combination thereof.

Oil-phase ingredients of creams can include, but are not limited to, dimethicone, dimethiconol, cyclomethicone, diisopropyl adipate, cetyl alcohol, stearyl alcohol, paraffin, petrolatum, almond oil, stearic acid, or a combination thereof. In particular aspects, aqueous ingredients can include, but are not limited to, purified water, glycerol (glycerin), propylene glycol, ethyl paraben, a humectant, or a combination thereof.

In some embodiments, the cream further comprises one or more film formers including but not limited to polyglycerylmethacrylate, acrylates/C₁₀-C₃₀ alkyl acrylate cross-polymers; antioxidant including but not limited to tocopheryl acetate; preservatives including but not limited to phenoxyethanol, benzyl alcohol; and/or other additives including but not limited to dicaprylyl ether, disodium EDTA, sodium hydroxide, and lactic acid.

Topically acceptable carrier lotions are typically liquid or semiliquid preparations in which solid particles are present in a water or alcohol base. Lotions are usually suspensions of solids, and can include a liquid oily emulsion of the oil-in-water type. Lotions are often desirable formulations because of the ease of applying a more fluid composition. It is generally advantageous for the insoluble matter in a lotion to be finely divided. Lotions will typically contain suspending agents to produce better dispersions as well as compounds useful for localizing and holding the Nerium oleander extract in contact with the skin, e.g., methylcellulose or sodium carboxymethylcellulose.

Non-limiting examples of topically acceptable carriers include hydrophilic cream base, hydrophilic lotion base, hydrophilic surfactant base, hydrophilic gel base, hydrophilic solution base, hydrophobic cream base, hydrophobic lotion base, hydrophobic surfactant base, hydrophobic gel base, hydrophobic solution base, and combinations thereof. Other non-limiting examples of topically acceptable carriers include cosmetic night cream, cosmetic day cream, foundation cream, suntan lotion, sunscreen, hand lotion, and combinations thereof. Additional non-limiting examples of topically acceptable carriers include surfactant, emulsifier, emulsion stabilizer, bulking agent, thickener, gellant, structuring agent, essential oil, emollient, moisturizer, pigment, colorant, anti-caking agent, anti-foaming agent, filler, film-forming agent, preservative, pH adjuster, water and combinations thereof. Further non-limiting examples of topically acceptable carriers include vitamin A, vitamin C, vitamin E, alpha-hydroxy or alpha-keto acids such as pyruvic acid, lactic acid and glycolic acid, lanolin, vaseline, methyl paraben, propyl paraben, mineral oil, vegetable oil, animal oil, organic and inorganic waxes, such as microcrystalline wax, paraffin wax and ozocerite wax, natural polymers, such as xanthanes, gelatin, cellulose, collagen, starch, and gum arabic, synthetic polymers such as polyethylene glycol (PEG), and combinations thereof.

The term “pharmaceutically acceptable carrier” as used herein has its ordinary meaning as understood by those skilled in the art and thus includes the topically acceptable carriers as described elsewhere herein that are useful for formulating pharmaceutical compositions. Non-limiting examples of pharmaceutically acceptable carriers include solutions, dispersions, emulsions, gels, creams, ointment, lotions, pastes, foams and combinations thereof. Pharmaceutical dosage forms can include sterile aqueous solutions or dispersions comprising the Nerium oleander extract adapted for the extemporaneous preparation of sterile solutions or dispersions, optionally encapsulated in liposomes. The ultimate dosage form should be fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, emu oil, nontoxic glyceryl esters, and suitable mixtures thereof. The proper fluidity of the composition can be maintained, for example, by the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride. Prolonged absorption of the compositions can be brought about by agents delaying absorption, for example, aluminum monostearate and/or gelatin.

The skin care products described herein may be formulated for any desired form of topical release, transdermal release or topical administration and/or transdermal administration of the Nerium oleander extract(s), including slow or delayed release preparations. Embodiments of skin care products may include known antioxidants (e.g., vitamin E); buffering agents; lubricants (e.g., synthetic or natural beeswax); sunscreens (e.g., para-aminobenzoic acid); and/or cosmetic agents (e.g., coloring agents, fragrances, essential oils, moisturizers, or drying agents).

An auxiliary agent such as casein, gelatin, albumin, or sodium alginate may also be included in various skin care products. Adjuvants such as fragrances and additional antimicrobial agents can be added to optimize the properties for a given use. Examples of fragrances include Ylang-Ylang oil, lavender oil, powder scent, jasmine, gardenia oil, or green tea oil. In addition, substances such as wetting or emulsifying agents, stabilizing agents, or pH buffering agents, may also be included. When a water-based carrier is used, the composition is typically near a neutral pH (+/−about 1, or 2, pH units).

The term “cosmetic treatment” as used herein has its ordinary meaning as understood by those skilled in the art and thus includes applying a cosmetic composition to skin in a manner that enhances appearance. For example, a cosmetic treatment may prevent or reduce the appearance of, and/or improve the appearance of, any one or more of the following: skin ageing; skin sagging; skin tone; skin firmness (e.g., improve appearance of wrinkles); elasticity of the skin; skin atrophy; density of the dermis and/or epidermis; volume of the dermis and epidermis; skin dehydration (e.g., reduce appearance of flaky skin); skin roughness; cellulite; development of adipose tissue in the hypodermis; glycation of molecules in the skin; symptoms of acne; skin breakdown due to the effects of oxidation; inflammatory conditions (e.g., reduce redness). A cosmetic treatment does not, and is not intended, to treat any medical condition or disorder.

The term “medical treatment” as used herein has its ordinary meaning as understood by those skilled in the art and thus includes applying a pharmaceutical composition to skin in a manner that is intended to treat a medical condition or disorder. As used herein, the terms “treat,” “treating,” “treatment,” “therapeutic,” and “therapy” do not necessarily mean total cure or abolition of the medical condition or disorder. Any alleviation of any undesired signs or symptoms of the medical condition or disorder, to any detectable extent can be considered treatment and/or therapy. Since the amounts of active ingredients used for medical treatment and typically greater than those used for cosmetic treatment, in many cases a medical treatment with also provide a cosmetic treatment. Furthermore, treatment may include acts that may worsen the subject's overall feeling of well-being or appearance.

The terms “effective amount” and “amount effective to treat” are used to indicate an amount of Nerium oleander extract(s) that elicits the cosmetic and/or medical response indicated. For example, a therapeutically effective amount of compound can be the amount needed to prevent, alleviate or ameliorate symptoms of the medical condition or disorder or prolong the survival of the subject being treated. This response may occur in a tissue, system, animal or human and includes alleviation of the signs or symptoms of the medical condition or disorder being treated. Determination of an effective amount for a particular medical and/or cosmetic treatment is well within the capability of those skilled in the art, in view of the disclosure provided herein. The effective amount of the active ingredient(s) disclosed herein required as a dose for a particular medical and/or cosmetic treatment will depend on the route of administration, the type of animal, including human, being treated, and the physical characteristics of the specific animal under consideration. The dose can be tailored to achieve a desired effect, but will depend on such factors as weight, diet, age, concurrent medication and other factors which those skilled in the medical and/or cosmetic arts will recognize.

“Subject” as used herein, means a human or a non-human mammal, e.g., a dog, a cat, a mouse, a rat, a cow, a sheep, a pig, a goat, a non-human primate or a bird.

An “effective amount” or a “pharmaceutically effective amount” as used herein refers to an amount of a therapeutic agent that is effective to relieve, to some extent, or to reduce the likelihood of onset of, one or more of the symptoms of a disease or condition, and can include (but does not require) curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a pharmaceutical composition in a manner effective for prophylactic and/or therapeutic purposes. The term “prophylactic treatment” refers to administering treatment to a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition to improve or stabilize the subject's condition.

### Methods of Extracting Nerium Oleander

Methods of extracting Nerium oleander plant material using various natural plant-based extractant liquids have now been developed. In some embodiments, an extraction method includes: 1) providing an extractant liquid that comprises a plant juice or plant oil; 2) intermixing a Nerium oleander plant species with the extractant liquid under conditions selected to form an extraction mixture; 3) conditioning the extraction mixture under conditions selected to extract the Nerium oleander plant species to thereby form a conditioned extraction mixture, wherein the conditioned extraction mixture comprises solid residual Nerium oleander plant species and a liquid Nerium oleander extract; and 4) separating at least a portion of the liquid Nerium oleander extract from the solid residual Nerium oleander plant species to thereby form a separated liquid Nerium oleander extract. U.S. Pat. No. 8,524,286 discloses oleandrin-containing extracts obtained by extracting Nerium oleander plant material with aloe. In contrast, the extractant liquids used in the Nerium oleander extraction methods described herein comprise at least one plant juice or plant oil that is not aloe. Thus, the extractant liquids used in the extraction methods described herein do not consist solely of aloe, nor do they consist solely of a mixture of aloe and an extraction adjuvant (such as an organic alcohol, ether, ketone or ester) that is not a plant juice or plant oil. Likewise, the separated liquid Nerium oleander extracts described herein do not consist solely of Nerium oleander aloe extracts, but instead (or in addition) comprise Nerium oleander plant juice and/or plant oil extracts that are not Nerium oleander aloe extracts.

In some embodiments, the intermixing of Nerium oleander plant species with the extractant liquid comprises mixing the extractant liquid with the leaves, stems and/or branches of a Nerium oleander plant species. Optionally, such leaves, stems and/or branches can be cut into pieces, milled, and/or powdered to facilitate the extraction.

In some embodiments, the extractant liquid comprises a plant oil. In some embodiments, the plant oil is avocado oil, banana oil, basil oil, canola oil, cocoa butter, corn oil, ginger oil, lavender oil, lemon oil, mint oil, olive oil, orange oil, parsley oil, rose oil, tea oil, vanilla oil, almond oil, cotton seed oil, hemp oil, linseed oil, neem oil, pecan oil, safflower oil, sesame oil, walnut oil, or any mixture of the foregoing.

In some embodiments, the plant oil is an herb oil. In some embodiments, the herb oil is a plant oil obtained from a plant of the Petroselinum genus or the Ocimum genus. The Petroselinum genus comprises two species, Petroselinum crispum (e.g., garden parsley) and Petroselinum segetum (e.g., corn parsley). The Ocimum genus comprises a number of species, including varieties of Ocimum basilicum (great basil or Saint Joseph's wort). In some embodiments, the plant oil is a basil oil. In some embodiments, the plant oil is a parsley oil.

In some embodiments, the extractant liquid is comprised of at least 50% of the plant oil. In some embodiments, the extractant liquid consists essentially of the plant oil. In other embodiments, the extractant liquid comprises a plant juice. In some embodiments, the extractant liquid is selected from the group consisting of cherry juice, grape juice, lemon juice, lime juice, orange juice, pear juice, pineapple juice, apple juice, cranberry juice, grapefruit juice. In some embodiments, the plant juice is a citrus juice. In some embodiments, the citrus juice is selected from the group consisting of lemon juice, lime juice and orange juice.

In some embodiments, the extractant liquid is comprised of at least 50% of the plant juice. In some embodiments, the extractant liquid consists essentially of the plant juice. In some embodiments, the extractant liquid comprises a plant oil and a plant juice. In some embodiments, the extraction mixture comprises an amount of Nerium oleander plant species in the range of about one part to about 10 parts by weight and an amount of plant oil or plant juice in the range of about one part to about 100 parts by weight, based on total weight of the extraction mixture.

An embodiment further provides conditioning the extraction mixture under conditions selected to extract Nerium oleander plant species to form a conditioned extraction mixture, wherein the conditioned extraction mixture comprises solid residual Nerium oleander plant species and a liquid Nerium oleander extract. In some embodiments, the conditioning comprises adding water to the extraction mixture. Examples of conditioning the extraction mixture under conditions selected to extract Nerium oleander plant species include heating the extraction mixture, agitating the extraction mixture, and heating the extraction mixture with agitation. In some embodiments, the conditioning comprises heating the extraction mixture to a temperature in the range of about 40° C. to about 100° C. to form the conditioned extraction mixture. In some embodiments, the conditioning comprises heating the extraction mixture for a heating time in the range of about 1 to about 10 hours.

Those skilled in the art will appreciate that the Nerium oleander extract may contain, in addition to oleandrin, other components extracted by the liquid extractant from the Nerium oleander plant species, such as, for example, other cardiac glycoside(s) and/or polysaccharide(s).

Various methods of separating the Nerium oleander extract from the solid residual Nerium oleander plant species may be used. For example, in various embodiments, separating at least a portion of the Nerium oleander extract from the solid residual Nerium oleander plant species may comprise filtration, centrifugation, flocculation and/or decanting.

Various other parameters of the extraction process such as, for example, time of heating, the manner in which the extract is heated and the size and number of filters which are utilized, can be modified.

### Pharmaceutical Compositions

Some embodiments relate to pharmaceutical composition, comprising a Nerium oleander extract as described herein, and a pharmaceutically acceptable carrier.

The pharmaceutical compositions described herein can be administered to a human patient per se, or in pharmaceutical compositions where they are mixed with other active ingredients, as in combination therapy, or suitable carriers or excipient(s). General techniques for formulation and administration of the Nerium oleander extract may be found in “Remington's Pharmaceutical Sciences,” Mack Publishing Co., Easton, Pa., 18th edition, 1990.

Suitable routes of application and/or administration may, for example, include topical (e.g., in the form a cosmetic composition or hair care product), oral, sublingual, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

The pharmaceutical compositions described herein may be manufactured in a manner that is itself known, e.g., by means of conventional mixing, dissolving, granulating, dragee-making, levigating, emulsifying, encapsulating, entrapping or tabletting processes.

Pharmaceutical compositions for use as described herein may be formulated in conventional manner using one or more physiologically acceptable carriers comprising excipients and auxiliaries which facilitate processing of the active compounds into preparations which can be used pharmaceutically. Proper formulation is dependent upon the intended use (e.g., cosmetic versus treatment of a disease or condition), and the route of application or administration chosen. Any of the well-known techniques, carriers, and excipients may be used as suitable and as understood in the art, e.g., as described in Remington's Pharmaceutical Sciences, above.

For injection, the Nerium oleander extract described herein may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the Nerium oleander extract described herein can be formulated readily by combining with pharmaceutically acceptable carriers well known in the art. Such carriers enable the extract described herein to be formulated as tablets, pills, sublingual compositions, dragees, capsules, liquids, gels, syrups, slurries, suspensions and the like, for oral ingestion by a patient to be treated. Pharmaceutical preparations for oral use can be obtained by mixing one or more solid excipients with the extract described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are typically provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the Nerium oleander extract in admixture with filler such as lactose, binders such as starches, and/or lubricants such as talc or magnesium stearate and, optionally, stabilizers. In soft capsules, the Nerium oleander extract described herein may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner. Alternatively, the Nerium oleander extract may be in powder form for constitution with a suitable vehicle, e.g., sterile pyrogen-free water, before use.

Other delivery systems for the Nerium oleander extract described herein may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the extract may be delivered using a sustained-release system. Various sustained-release materials have been established and are well known by those skilled in the art. Sustained-release capsules may, depending on their chemical nature, extend the release of the Nerium oleander extract for a few hours up to a few weeks.

Pharmaceutical compositions suitable for use include compositions where the Nerium oleander extract described herein is contained in an amount effective to achieve its intended purpose. More specifically, a therapeutically effective amount means an amount of the extract described herein effective to prevent, alleviate or ameliorate symptoms of disease or prolong the survival of the subject being treated. Determination of a therapeutically effective amount is well within the capability of those skilled in the art, especially in light of the detailed disclosure provided herein.

### Cosmetic Compositions

Some embodiments relate to a cosmetic composition, comprising the Nerium oleander extract described herein, and a topically acceptable carrier. Such compositions can be prepared by combining the Nerium oleander extract with the topically acceptable carrier in the manner described elsewhere herein, e.g., to form a solution, dispersion, emulsion, gel, cream, ointment, lotion, paste, or foam, that is suitable for topical administration in an amount and manner which provides a cosmetic treatment.

### Treatment of Skin Conditions

Some embodiments relate to a method of treatment, comprising identifying a subject having a skin condition; and applying an effective amount of a pharmaceutical composition described herein to the skin of the subject to thereby treat the skin condition.

It is to be understood that, as used herein, the terms treating and treatment include and encompass preventing, reducing, ameliorating, improving, alleviating, and/or eliminating the dermatological effects of aging, with particular regard to loss of subcutaneous fat, and in particular, facial fat loss and sagging skin. The pharmaceutical compositions and methods described herein are also suitable for use in treating various cancers, such as skin cancers, as well as other cancers as described in Newman, Robert A. et al. “Cardiac Glycosides as Novel Cancer Therapeutic Agents”, Molecular Interventions Vol. 8, 36-49 (2008). In preferred embodiments, the pharmaceutical compositions are applied to the face.

The present compositions alleviate symptoms or conditions, including, but not limited to: loss of subcutaneous fat, loss of facial fat, sagging skin, aging skin, wrinkles, dry skin, lack of skin tone, acne, uneven skin tone, age spots, liver spots, burns, sunburn, heat burn, radiation burn, firmness, or plumpness, and/or thinning skin.

In accordance with this invention the extracts comprise compositions which include, without limitation, topically applied sunscreens, anti-oxidants, anti-inflammatories, cosmetics, including makeups, anti-aging formulations, e.g., creams for fine lines and/or wrinkles, topicals, skin permeants, and the like. Also in accordance with this invention, ingredients, components, or compounds that are formulated in such compositions in a variety of product forms, e.g., transdermals, such as patches, and the like, are encompassed, particularly for topical administration.

Another aspect of the present invention provides the compositions comprising the extracts preferably for topical administration without inducing significant irritation. Further, such compositions are preferably delivered by, but not limited to, the use of targeted delivery systems, for example, liposomes, microspheres, transdermal patches, and the like, so that the active agents can more readily penetrate the skin layer of the area of application, e.g., face or neck, or the subcutaneous layer of the skin, where adipocytes (i.e. fat cells) are located. Compositions comprising plant constituents or plant extract are preferably administered topically.

### Cosmetic Treatments

Some embodiments relate to a method of applying a cosmetic, comprising identifying a subject in need of cosmetic application; and applying a cosmetically effective amount of a cosmetic composition described herein to the skin of the subject.

### EXAMPLES

Additional embodiments are disclosed in further detail in the following examples, which are not in any way intended to limit the scope of the claims.

### Example 1

Parsley oil (500 g) is mixed with a 100 g of a Nerium oleander plant powder (milled product of dried leaves and stems of a Nerium oleander plant) to form an extraction mixture. The extraction mixture is conditioned by stirring and heating at 70-80° C. for four hours to form a conditioned extraction mixture. After cooling to ambient temperature, the conditioned extraction mixture is filtered through a coarse filter to separate the residual plant solids from the liquid. The resulting separated liquid, which is a parsley oil Nerium oleander extract, is filtered through a medium filter to yield a clarified extract.

### Example 2

Basil oil (400 g) is mixed with a 100 g of a Nerium oleander plant powder (ground product of the dried leaves and stems of a Nerium oleander plant) to form an extraction mixture. The extraction mixture is conditioned by stirring and heating at 75-85° C. for three hours to form a conditioned extraction mixture. After cooling to ambient temperature, the conditioned extraction mixture is filtered through a coarse filter and a medium filter to separate the residual plant solids from the liquid. The resulting separated liquid is a clarified basil oil Nerium oleander extract.

### Example 3

Orange juice (600 g) is mixed with 100 g of a Nerium oleander plant powder (ground product of the dried leaves and stems of a Nerium oleander plant) to form an extraction mixture. The extraction mixture is conditioned by stirring and heating at 65-75° C. for five hours to form a conditioned extraction mixture. After cooling to about 30° C., the conditioned extraction mixture is filtered through a medium filter to separate the residual plant solids from the liquid. The resulting separated liquid is an orange juice Nerium oleander extract.

### Example 4

Apple juice (240 g) is mixed with 50 g of a Nerium oleander plant powder (ground product of the dried leaves and stems of a Nerium oleander plant) to form an extraction mixture. The extraction mixture is conditioned by stirring and heating at 60-70° C. for six hours to form a conditioned extraction mixture. After cooling to ambient temperature, the conditioned extraction mixture is cooled and filtered through a coarse filter and a fine filter to separate the residual plant solids from the liquid. The resulting separated liquid is a clear apple juice Nerium oleander extract.

### Examples 5-8

Four batches of cosmetic compositions are prepared by combining each of the Nerium oleander extracts of Examples 1-4 with a cosmetically acceptable carrier. The weight ratio of Nerium oleander extract to carrier for each batch is about 1 to 10. The carrier contains glycerin, glyceryl stearate, PEG-200 Stearate, cetyl alcohol, dimethicone, hydroxymethylcellulose, methyl paraben, vitamin E, and fragrance. Each of the four cosmetic compositions is applied to the face and hands of a human subject to alleviate the appearance of aging skin.

### Example 9-12

Four batches of pharmaceutical compositions are prepared by combining each of the Nerium oleander extracts of Examples 1-4 with a pharmaceutically acceptable carrier. The weight ratio of Nerium oleander extract to carrier for each batch is about 3 to 1. The carrier contains isostearic acid, cetyl alcohol, stearyl alcohol, white petrolatum, polysorbate 60, sorbitan monostearate, glycerin, xanthan gum, purified water, benzyl alcohol, methylparaben, and propylparaben. Each of the four pharmaceutical compositions is applied to the face and hands of a human subject to alleviate the symptoms of skin cancer.

1. A method of extracting Nerium oleander, comprising:
providing an extractant liquid that comprises a plant juice or plant oil;
intermixing a Nerium oleander plant species with the extractant liquid under conditions selected to form an extraction mixture;
conditioning the extraction mixture under conditions selected to extract the Nerium oleander plant species to thereby form a conditioned extraction mixture, wherein the conditioned extraction mixture comprises solid residual Nerium oleander plant species and a liquid Nerium oleander extract; and
separating at least a portion of the liquid Nerium oleander extract from the solid residual Nerium oleander plant species to thereby form a separated liquid Nerium oleander extract;
wherein the extractant liquid does not consist of aloe.

2. The method of claim 1, wherein the extractant liquid comprises the plant oil.

3. The method of claim 2, wherein the plant oil is selected from the group consisting of avocado oil, banana oil, basil oil, canola oil, cocoa butter, corn oil, ginger oil, lavender oil, lemon oil, mint oil, olive oil, orange oil, parsley oil, rose oil, tea oil, vanilla oil, almond oil, cotton seed oil, hemp oil, linseed oil, neem oil, pecan oil, safflower oil, sesame oil, walnut oil, and any mixture of the foregoing.

4. The method of claim 2, wherein the plant oil is an herb oil.

5. The method of claim 4, wherein the herb oil is a plant oil of a plant in the Petroselinum genus or the Ocimum genus.

6. The method of claim 4, wherein the plant oil is a basil oil.

7. The method of claim 4, wherein the plant oil is a parsley oil.

8. The method of claim 2, wherein the extractant liquid is comprised of at least 50% of the plant oil.

9. The method of claim 2, wherein the extractant liquid consists essentially of the plant oil.

10. The method of claim 1, wherein tag extractant liquid comprises the plant juice.

11. The method of claim 10, wherein the extractant liquid is selected from the group consisting of cherry juice, grape juice, lemon juice, lime juice, orange juice, pear juice, pineapple juice, apple juice, cranberry juice, grapefruit juice, and any mixture of the foregoing.

12. The method of claim 10, wherein the plant juice is a citrus juice.

13. The method of claim 12, wherein the citrus juice is selected from the group consisting of lemon juice, lime juice and orange juice.

14. The method of claim 10, wherein the extractant liquid is comprised of at least 50% of the plant juice.

15. The method of claim 10, wherein the extractant liquid consists essentially of the plant juice.

16. The method of claim 1, wherein said conditioning comprises heating said extraction mixture to a temperature in the range of about 40° C. to about 100° C. to form said conditioned extraction mixture.

17. The method of claim 1, wherein said conditioning comprises heating said extraction mixture for a heating time in the range of about 1 to about 10 hours.

18. The method of claim 1, wherein said separating comprises subjecting the conditioned extraction mixture to a separation method selected from the group consisting from filtration, centrifugation, and decanting.

19. The method of claim 1, wherein said conditioning comprising adding water to said extraction mixture.

20. A liquid Nerium oleander extract prepared by the method of claim 1.

21. A pharmaceutical composition, comprising the Nerium oleander extract prepared according to the method of claim 1, and a pharmaceutically acceptable carrier.

22. A cosmetic composition, comprising the Nerium oleander extract prepared according to the method of claim 1, and a cosmetically acceptable carrier.

23. A method of treatment, comprising identifying a subject having a skin condition; and applying an effective amount of the pharmaceutical composition of claim 21 to the skin of the subject to thereby treat the skin condition.

24. A method of applying a cosmetic, comprising identifying a subject in need of cosmetic application; and applying a cosmetically effective amount of the cosmetic composition of claim 22 to the skin of the subject.
